(19) United States
(12) Patent Application Publication
(10) Pub. No.: US 2009/0235474 A1
(43) Pub. Date: Sep. 24, 2009

(54) COMBINATION TOOTHBRUSH AND TONGUE CLEANER

(76) Inventor: Jon Seigel, Playa Vista, CA (US)

Correspondence Address: BELASCO, JACOBS & TOWNSLEY LLP;HOWARD HUGHES CENTER, 6701 CENTER DRIVE WEST, 14th Floor, LOS ANGELES, CA, 90045, US

(21) Appl. No.: 12/052,557

(22) Filed: Mar. 20, 2008

(51) Int. Cl. A46B 15/00; A61B 17/24

(52) U.S. Cl. 15/111; 15/167.1; 15/143.1; 606/161

(57) ABSTRACT

A combination toothbrush and tongue cleaner includes handle that has a brush portion, a gripping portion and a tongue cleaner portion. A brush element is located adjacent an end of the brush portion and includes a plurality of bristles affixed orthogonally to the brush portion. A tongue cleaner element is located adjacent an end of the tongue cleaner portion. It is affixed orthogonally to the tongue cleaner portion and located orthogonally to the brush element. The brush portion and tongue cleaner portion are each angled upwardly from the gripping portion. The tongue cleaner element has a scraping edge that has first and second ends and is located above an upper surface of the tongue cleaner portion. The scrapping edge is located at an angle to the tongue cleaner portion. The scrapping edge is curved toward the brush element. The scrapping edge has inward curving tip segments located at the ends.

### FIELD OF INVENTION

This invention relates to the field of oral cleaning devices, and more specifically to a combination toothbrush and tongue cleaner.

### BACKGROUND OF THE INVENTION

Modern dental practice has determined that to properly clean the month and to reduce the incidence of halitosis it is necessary to clean not only the teeth, but the tongue as well. Various techniques have been developed for effecting such cleaning, including toothbrushes, dental floss, mouthwash, and tongue scrapers. In order to reduce the need to have multiple tools for effecting oral cleaning, particularly when traveling, various combination devices have been developed. The following are several examples.

U.S. Patent Application No. 2004/0249402, published for Von Bergen, discloses a low profile tongue scraper that includes a conventional toothbrush at one end and a scraping element at the other end. Both ends of the device, one to the brush and one to the scraper, are angled. The scraping element is transverse to the handle.

U.S. Pat. No. 5,915,433, issued to Hansen is directed to the ornamental design of a pair of trunks. The trunks have an attached skirt that covers the front and the rear, but not the sides. U.S. Pat. No. 5,915,433

U.S. Pat. No. 5,915,433, issued to Hybler a combined toothbrush and tongue scraper with anti-slip bumps between the toothbrush portion and the scraper portion. The device is seen to have a tooth brushing end at the opposite end of the device wherein is found a tongue scraping portion that is transverse to the elongated plastic base portion. On the first tongue scraping end and the second tooth brushing end, there is an intermediate handle portion with a plurality of bumps that are used to preclude slipping of the device in a user's hand.

U.S. Pat. No. D377,417, issued to Gupta disclose a tongue cleaner integrally molded with a toothbrush. The tongue scraping element is angled to the main body of the instrument.

U.S. Pat. No. 5,842,247, issued to Decesare is directed to a combination toothbrush and tongue brush. The tongue brush portion includes a curvilinear shaft integrally coupled with the second end of the main body portion of the device. The combination toothbrush and tongue brush also includes a plurality of gripping ridges which are integrally formed and extend upwardly from an exterior surface of the main body portion intermediate first and second ends of the device.

U.S. Pat. No. D447,238, issued to Tang discloses a tongue scraping tool in which the tongue scraping end of the device is angled from the main portions of the handle.

It is an objective of the present invention to provide a compact device that combines a toothbrush with a tongue cleaning feature. It is a further objective to provide such a device that fits easily in the hand and is convenient to use. It is yet a further objective to provide a tooth and tongue cleaning device that is convenient for travel use while providing effective and rapid cleaning. Finally, it is an objective of the present invention to provide such device that is durable and inexpensive to manufacture.

While some of the objectives of the present invention are disclosed in the prior art, none of the inventions found include all of the requirements identified.

### SUMMARY OF THE INVENTION

The present invention addresses all of the deficiencies of prior art combination toothbrush and tongue cleaner inventions and satisfies all of the objectives described above.

(1) A combination toothbrush and tongue cleaner may be constructed from the following components. A handle is provided. The handle has a brush portion, a gripping portion and a tongue cleaner portion. A brush element is provided. The brush element is located adjacent an end of the brush portion and has a plurality of bristles affixed orthogonally to the brush portion. A tongue cleaner element is provided. The tongue cleaner element is located adjacent an end of the tongue cleaner portion. It is affixed orthogonally to the tongue cleaner portion and located orthogonally to the brush element. The brush portion is angled upwardly from the gripping portion. The tongue cleaner portion is angled upwardly from the gripping portion.

(2) In a variant of the invention, the tongue cleaner element has a scraping edge. The scraping edge has first and second ends and is located above an upper surface of the tongue cleaner portion.

(3) In another variant, the scrapping edge is located at an angle to the tongue cleaner portion.

(4) In still another variant, the scrapping edge is curved toward the brush element.

(5) In yet another variant, the scrapping edge has inward curving tip segments located at the first and second ends.

(6) In a further variant, the scrapping edge is beveled at a back edge toward a front edge. The front edge faces toward the brush element.

(7) In still a further variant, the gripping portion further includes grip enhancing features.

(8) In yet a further variant, the grip enhancing features are selected from the group consisting of finger grooves, indentations, serrations and raised projections.

(9) In a final variant of the invention, a finger pad is included. The finger pad has a frictional surface and an attachment surface. The finger pad is affixed at the attachment surface to a back side of the tongue cleaner portion between the tongue cleaner element and the gripping portion. The frictional surface permits application of finger pressure to urge the tongue cleaner element against a user's tongue.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a perspective view of the preferred embodiment of the invention illustrating the curved tongue cleaner;

FIG. 2 is a plan view of the FIG. 1 embodiment;

FIG. 2A is a plan view detail of a tongue cleaner with curved in ends;

FIG. 3 is a side elevational view of the FIG. 1 embodiment illustrating raised projections in the gripping portion;

FIG. 3A is a side elevational detail view of a cleaning element with a curved scraping edge;

FIG. 3B is a side elevational detail view of a cleaning element with a flat scraping edge;

FIG. 4 is a bottom view of the FIG. 1 embodiment illustrating the finger pad;

FIG. 5 is a side elevational view of the FIG. 1 embodiment illustrating an indentation in the gripping portion.

### DETAILED DESCRIPTION OF THE PREFERRED EMBODIMENT

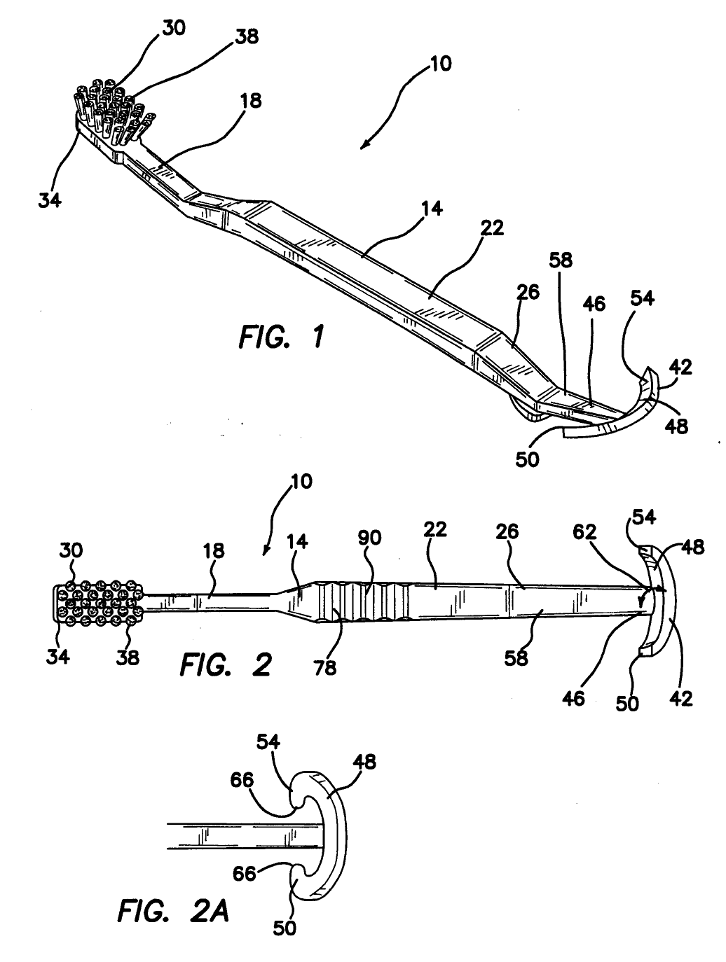

(1) FIGS. 1-5 illustrate a combination toothbrush and tongue cleaner 10 that may be constructed from the following components. A handle 14 is provided. The handle 14 has a brush portion 18, a gripping portion 22 and a tongue cleaner portion 26. A brush element 30 is provided. The brush element 30 is located adjacent an end 34 of the brush portion 18 and has a plurality of bristles 38 affixed orthogonally to the brush portion 18. A tongue cleaner element 42 is provided. The tongue cleaner element 42 is located adjacent an end 46 of the tongue cleaner portion 26. It is affixed orthogonally to the tongue cleaner portion 26 and located orthogonally to the brush element 30. The brush portion 18 is angled upwardly from the gripping portion 22. The tongue cleaner portion 26 is angled upwardly from the gripping portion 22.

(2) In a variant of the invention, the tongue cleaner element 42 has a scraping edge 48. The scraping edge 48 has first 50 and second 54 ends and is located above an upper surface 58 of the tongue cleaner portion 26.

(3) In another variant, the scrapping edge 48 is located at an angle 62 to the tongue cleaner portion 26.

(4) In still another variant, the scrapping edge 48 is curved toward the brush element 30.

(5) In yet another variant, as illustrated in FIG. 2A, the scrapping edge 48 has inward curving tip segments 66 located at the first 50 and second 54 ends.

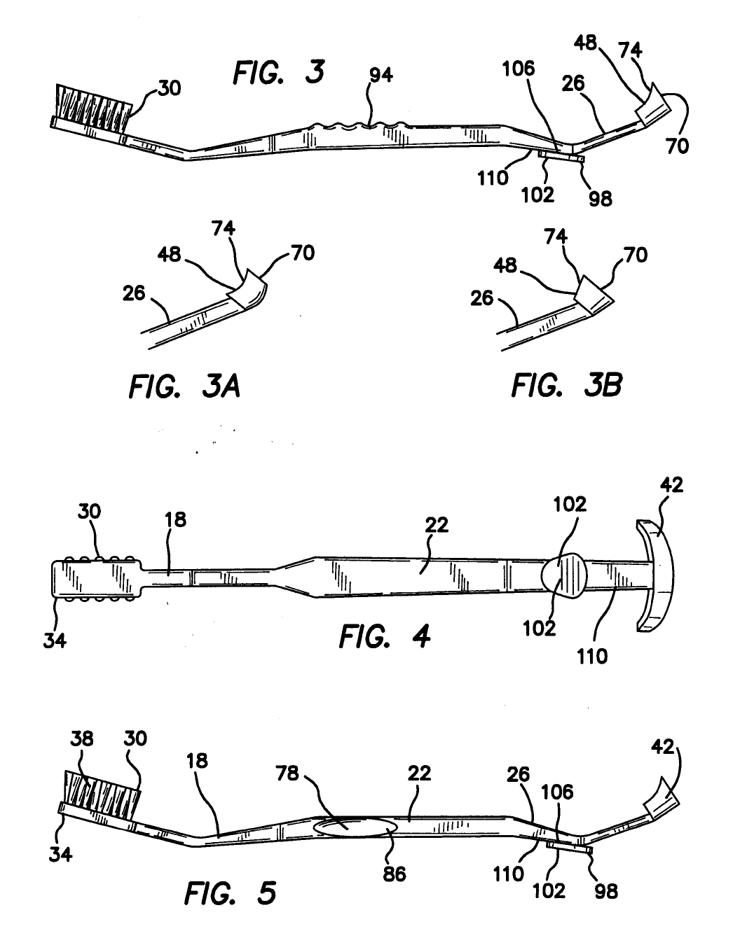

(6) In a further variant, as illustrated in FIGS. 3, 3A and 3B, the scrapping edge 48 is beveled at a back edge 70 toward a front edge 74. The front edge 74 faces toward the brush element 30.

(7) In still a further variant, as illustrated in FIGS. 2, 3 and 5, the gripping portion 22 further includes grip enhancing features 78.

(8) In yet a further variant, the grip enhancing features 78 are selected from the group consisting of finger grooves (not shown), indentations 86,.serrations 90 and raised projections 94.

(9) In a final variant of the invention, as illustrated in FIGS. 3-5, a finger pad 98 is included. The finger pad 98 has a frictional surface 102 and an attachment surface 106. The finger pad 98 is affixed at the attachment surface 106 to a back side 110 of the tongue cleaner portion 26 between the tongue cleaner element 42 and the gripping portion 22. The frictional surface 102 permits application of finger pressure to urge the tongue cleaner element 42 against a user's tongue (not shown).

The combination toothbrush and tongue cleaner 10 has been described with reference to particular embodiments. Other modifications and enhancements can be made without departing from the spirit and scope of the claims that follow.

1. A combination toothbrush and tongue cleaner, comprising:
a handle, said handle having a brush portion, a gripping portion and a tongue cleaner portion;
a brush element, said brush element being disposed adjacent an end of said brush portion and having a plurality of bristles affixed orthogonally to said brush portion;
a tongue cleaner element, said tongue cleaner element being disposed adjacent an end of said tongue cleaner portion and being affixed orthogonally to said tongue cleaner portion and disposed orthogonally to said brush element;
said brush portion angled upwardly from said gripping portion; and
said tongue cleaner portion angled upwardly from said gripping portion.

2. The combination toothbrush and tongue cleaner, as described in claim 1, wherein said tongue cleaner element has a scraping edge, said scraping edge having first and second ends and being disposed above an upper surface of said tongue cleaner portion.

3. The combination toothbrush and tongue cleaner, as described in claim 2, wherein said scrapping edge is disposed at an angle to said tongue cleaner portion.

4. The combination toothbrush and tongue cleaner, as described in claim 2, wherein said scrapping edge is curved toward said brush element.

5. The combination toothbrush and tongue cleaner, as described in claim 2, wherein said scrapping edge has inward curving tip segments disposed at said first and second ends.

6. The combination toothbrush and tongue cleaner, as described in claim 2, wherein said scrapping edge is beveled at a back edge toward a front edge, said front edge facing toward said brush element.

7. The combination toothbrush and tongue cleaner, as described in claim 1, wherein said gripping portion further comprises grip enhancing features.

8. The combination toothbrush and tongue cleaner, as described in claim 7, wherein said grip enhancing features are selected from the group consisting of:
finger grooves, indentations, serrations and raised projections.

9. The combination toothbrush and tongue cleaner, as described in claim 1, further comprising a finger pad:
said finger pad having a frictional surface and an attachment surface;
said finger pad being affixed at said attachment surface to a back side of said tongue cleaner portion between said tongue cleaner element and said gripping portion; and
said frictional surface permitting application of finger pressure to urge said tongue cleaner element against a user's tongue.
